(19) United States
(12) Patent Application Publication
(10) Pub. No.: US 2017/0143309 A1
(43) Pub. Date: May 25, 2017

(54) ULTRASONIC PROBE, CONTROL DEVICE, AND MEASUREMENT APPARATUS

(71) Applicant: SEIKO EPSON CORPORATION, Tokyo (JP)

(72) Inventors: Ryutaro SEKI, Suwa-shi (JP); Hiromitsu MIZUKAMI, Matsumoto-shi (JP)

(73) Assignee: SEIKO EPSON CORPORATION, Tokyo (JP)

(21) Appl. No.: 15/360,961

(22) Filed: Nov. 23, 2016

(30) Foreign Application Priority Data
Nov. 25, 2015 (JP) 2015-229830

(51) Int. Cl. A61B 8/00; A61B 8/04; A61B 8/08; A61B 8/02; A61B 8/06

(52) U.S. Cl. CPC A61B 8/4494; A61B 8/54; A61B 8/02; A61B 8/06; A61B 8/5223; A61B 8/04; A61B 8/488; A61B 8/0891

(57) ABSTRACT

An ultrasonic probe includes ultrasonic transducer arrays of N types (where N≧2) for which drive frequencies are different from each other, and a common signal line that is used for both transmission and reception and to which the ultrasonic transducer arrays are connected in common.

### BACKGROUND

1. Technical Field

The present invention relates to an ultrasonic probe including an ultrasonic transducer array, and the like.

2. Related Art

Biological information (hereinafter, referred to as “vasculature biological information”) regarding a blood vessel system which circulates blood includes various pieces of information such as blood pressure, a blood vessel diameter, pulse wave propagation velocity, a blood flow rate, and a thickness of the intima and the media. There are various methods of measuring the vasculature biological information, and, above all, there is an ultrasonic measurement method in which ultrasonic waves are transmitted to and received from blood vessels as a noninvasive method (for example, JP-A-2005-52424).

However, in the ultrasonic measurement of the related art, generally, an ultrasonic probe including one or a plurality of ultrasonic transducer arrays (hereinafter, abbreviated to “ultrasonic TD array”) for which drive frequencies are the same as each other is used.

Not only in order to improve measurement accuracy but also in order to measure pulse wave propagation velocity, there is a case where ultrasonic measurement is desired to be performed at the same time or with a slight time difference (hereinafter, collectively referred to as “the same time”) at a plurality of locations. However, in the ultrasonic probe of the related art, since drive frequencies for respective ultrasonic TD arrays are the same as each other, a gap between the ultrasonic TD arrays (that is, a gap between measurement locations) is required to be increased as much as possible in order to prevent interference between transmitted waves which are transmitted through a living body or to prevent interference between reflected waves (received waves).

### SUMMARY

An advantage of some aspects of the invention is to propose a new technique capable of realizing transmission and reception of ultrasonic waves in parallel at a plurality of locations in ultrasonic measurement.

A first aspect of the invention is directed to an ultrasonic probe including ultrasonic transducer arrays of N types (where N≧2) for which drive frequencies are different from each other; and a common signal line that is used for both transmission and reception and to which the ultrasonic transducer arrays are connected in common.

According to the first aspect, the ultrasonic probe includes ultrasonic transducer arrays of N types for which drive frequencies are different from each other. Thus, it is possible to prevent the occurrence of interference in transmission and reception of ultrasonic waves. Therefore, it is possible to perform transmission and reception in parallel at N locations. The ultrasonic transducer arrays of N types are connected in common to the common signal line which is used for both transmission and reception. Consequently, it is possible to reduce the number of signal cables.

As a second aspect, the first aspect may be configured as the ultrasonic probe in which the ultrasonic transducer arrays of N types are provided to be parallel to each other in a column.

According to the second aspect, since the ultrasonic transducer arrays of N types are provided to be parallel to each other in a column, it is possible to implement a configuration suitable for performing ultrasonic measurement on the same blood vessel as a measurement target at different positions.

As a third aspect, the second aspect may be configured as the ultrasonic probe in which a gap between the ultrasonic transducer arrays which are adjacent to each other is 1 cm or more and 10 cm or less.

According to the third aspect, it is possible to implement the ultrasonic probe in which a gap between the ultrasonic transducer arrays is 1 cm or more and 10 cm or less.

As a fourth aspect, the second or third aspect may be configured as the ultrasonic probe in which a gap between the ultrasonic transducer arrays is configured to be changed.

According to the fourth aspect, it is possible to implement the ultrasonic probe provided with a mechanism changing a gap between the ultrasonic transducer arrays.

As a fifth aspect, any one of the first to fourth aspects may be configured as the ultrasonic probe in which, in the ultrasonic transducer arrays of N types, an immediately higher drive frequency is twice or more higher than an immediately lower drive frequency in the order starting from the highest drive frequency.

According to the fifth aspect, in the ultrasonic transducer arrays of N types, an immediately higher drive frequency is twice or more higher than an immediately lower drive frequency in the order starting from the highest drive frequency. Therefore, it is possible to effectively prevent interference when the ultrasonic transducer arrays of N types transmit and receive ultrasonic waves.

A sixth aspect of the invention is directed to a control device controlling the ultrasonic probe according to anyone of the first to fifth aspects, including a transmission controller that generates a drive signal for driving the ultrasonic transducer arrays of N types, and outputs the drive signal to the common signal line; and a reception controller that receives a mixed received signal from the common signal line, and extracts an individual received signal for each of the ultrasonic transducer arrays.

According to the sixth aspect, it is possible to implement the control device controlling transmission and reception of ultrasonic waves in the ultrasonic probe of the first to fifth aspects.

As a seventh aspect, the sixth aspect may be configured as the control device in which the transmission controller generates the drive signal as a signal including drive waveforms corresponding to drive frequencies for the respective ultrasonic transducer arrays of N types in a time division manner.

According to the seventh aspect, the drive signal which is output to the common signal line may be a signal including drive waveforms corresponding to drive frequencies for the respective ultrasonic transducer arrays of N types in a time division manner.

According to an eighth aspect, the sixth or seventh aspect may be configured as the control device in which the reception controller includes a filter unit corresponding to a drive frequency for each of the ultrasonic transducer arrays of N types.

Signals received by the respective ultrasonic transducer arrays of N types are mixed and carried through the common signal line. However, according to the eighth aspect, since the filter unit corresponding to the drive frequency of each ultrasonic transducer array is provided, an individual received signal received by each ultrasonic transducer array can be extracted.

A ninth aspect of the invention is directed to a measurement apparatus including the ultrasonic probe according to any one of the first to fifth aspects that performs transmission and reception of ultrasonic waves toward the same blood vessel of a subject in parallel; and the control device according to any one of the sixth to eighth aspects, in which the measurement apparatus measures vasculature biological information.

According to the ninth aspect, it is possible to implement the apparatus which performs transmission and reception of ultrasonic waves toward the same blood vessel of a subject in parallel so as to measure vasculature biological information.

More specifically, as a tenth aspect, the ninth aspect may be configured as the measurement apparatus in which at least one of pulse wave propagation velocity, a blood vessel diameter, and a blood flow rate is measured as the vasculature biological information.

As an eleventh aspect, the ninth aspect may be configured as the measurement apparatus which further includes a relative blood vessel position determination unit that determines relative blood vessel positions which are blood vessel positions as measurement targets in the respective ultrasonic transducer arrays, on the basis of the individual received signal; a propagation length calculation unit that calculates a propagation length of a pulse wave along a blood vessel by using a gap between the adjacent ultrasonic transducer arrays and a gap between the relative blood vessel positions; and a pulse wave propagation velocity calculation unit that calculates pulse wave propagation velocity by using the individual received signal and the propagation length.

### BRIEF DESCRIPTION OF THE DRAWINGS

The invention will be described with reference to the accompanying drawings, wherein like numbers reference like elements.

FIG. 1 is a diagram for explaining the entire configuration of a measurement apparatus.

FIG. 2 is a schematic diagram of a rear surface side of an ultrasonic probe.

FIG. 3 is a sectional view at a position where the ultrasonic probe is attached.

FIG. 4 is a diagram illustrating time-series waveform examples of a first blood vessel diameter and a second blood vessel diameter.

FIG. 5 is a diagram illustrating acceleration waveforms obtained by performing second order differentiation on the blood vessel diameters illustrated in FIG. 4.

FIG. 6 is a diagram illustrating an example of a waveform of a drive signal.

FIG. 7 is a diagram for explaining a configuration of a control device related to transmission and reception of ultrasonic waves.

FIG. 8 is a diagram for explaining a configuration of the control device related to transmission and reception of ultrasonic waves.

FIG. 9 is a block diagram illustrating a functional configuration example of the measurement apparatus.

FIG. 10 is a diagram illustrating a configuration example of blood vessel diameter log data.

FIG. 11 is a diagram illustrating a configuration example of pulse wave propagation velocity log data.

FIG. 12 is a flowchart for explaining a flow of principal processes in the measurement apparatus.

FIG. 13 is a diagram illustrating an example of a drive signal for ultrasonic TD arrays of three types.

FIG. 14 is a diagram illustrating an example of a configuration in which the ultrasonic TD arrays of three types are disposed.

FIG. 15 is a diagram illustrating an example of a mechanism which changes a gap between the ultrasonic TD arrays.

FIG. 16 is a diagram illustrating an example of a mechanism which changes a gap between the ultrasonic TD arrays.

### DESCRIPTION OF EXEMPLARY EMBODIMENTS

Hereinafter, an embodiment to which the invention is applied will be described, but, of course, an applicable form of the invention is not limited to the following embodiment.

### Entire Outline

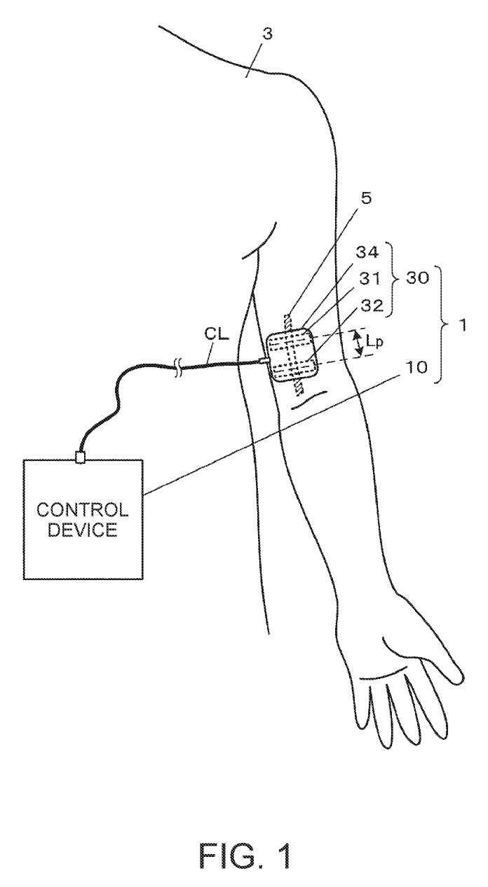

FIG. 1 is a diagram for explaining the entire configuration of a measurement apparatus 1 in the present embodiment, and is a diagram illustrating a state in which an ultrasonic probe 30 is attached.

The measurement apparatus 1 has a configuration in which a control device 10 is electrically connected to the ultrasonic probe 30 via a common signal line CL, and is a device which measures biological information (hereinafter, referred to as “vasculature biological information”) regarding a blood vessel system circulating blood, by using ultrasonic waves. In the present embodiment, finally measured vasculature biological information is pulse wave propagation velocity, and other vasculature biological information such as a blood vessel diameter is measured in the process thereof.

The control device 10 is a kind of computer, and controls the ultrasonic probe 30 so as to measure a blood vessel diameter of a blood vessel 5 in real time. The pulse wave propagation velocity is calculated on the basis of a measurement result. The pulse wave propagation velocity may be calculated for each beat.

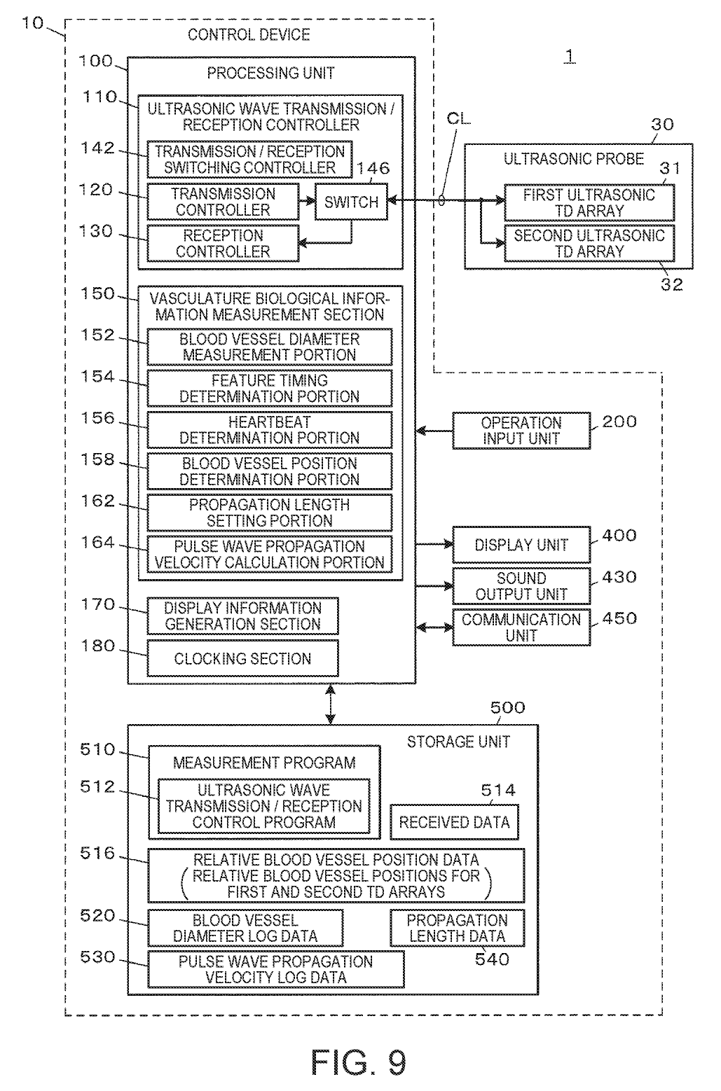

As a specific functional configuration of the control device 10 will be described later with reference to FIG. 9, the control device 10 is configured to include a processing unit 100 which has a processor or the like and performs various calculation controls; an operation input unit 200 which is provided with a touch panel or buttons and via which a user inputs an operation; a display unit 400 which is provided with a liquid crystal screen or the like and displays various pieces of information for a user; a sound output unit 430 such as a speaker; a communication unit 450 which performs communication with an external apparatus; a storage unit 500 which is provided with a hard disk or a memory and stores a program or data; and the like.

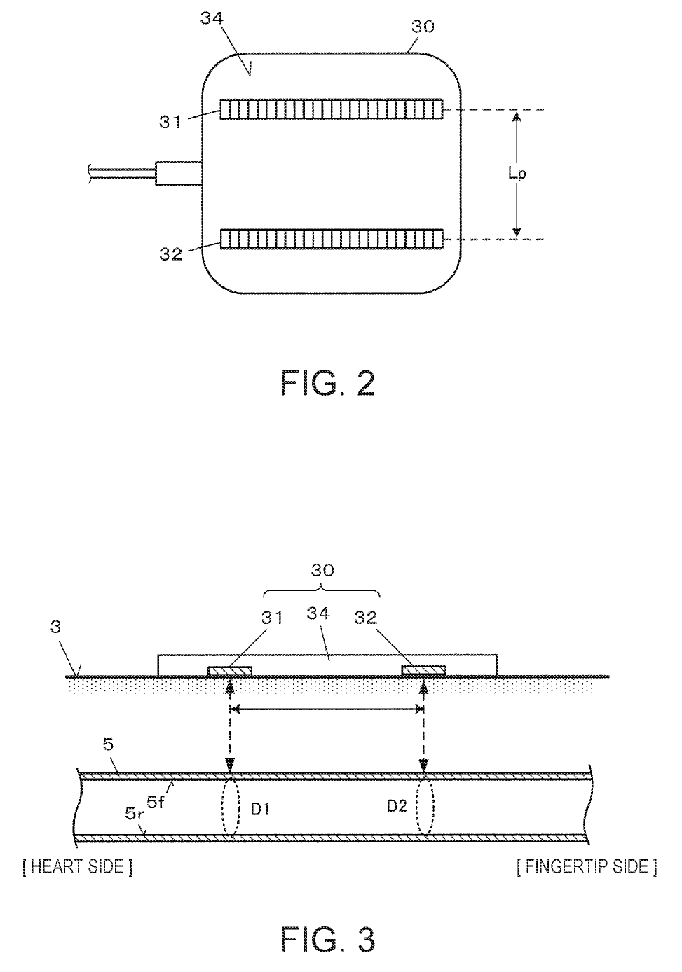

The ultrasonic probe 30 includes a first ultrasonic TD array 31 as an ultrasonic transducer array (hereinafter, abbreviated to “ultrasonic TD array”) which is driven at a first drive frequency, and a second ultrasonic TD array 32 which is driven at a second drive frequency, and a thin probe which is attached to the skin of a subject 3. FIG. 2 is a schematic diagram of a rear surface (an attachment surface on which the ultrasonic probe is attached to the skin of the subject 3) side of the ultrasonic probe 30. Each of the first ultrasonic TD array 31 and the second ultrasonic TD array 32 is provided with a plurality of arranged ultrasonic transducer elements, and transmits or applies ultrasonic pulses to the subject 3 and receives reflected waves thereof. In the present embodiment, as illustrated in FIG. 2, each of the first ultrasonic TD array 31 and the second ultrasonic TD array 32 is configured by linearly disposing the plurality of transducer elements, but may be configured by disposing the plurality of transducer elements in a two-dimensional manner.

The first drive frequency for driving the first ultrasonic TD array 31 is different from the second drive frequency for driving the second ultrasonic TD array 32, and a higher (higher-level) drive frequency is twice or more higher than a lower (lower-level) drive frequency. If the drive frequencies are different from each other, it is possible to prevent the occurrence of mutual interference in transmission and reception of ultrasonic waves in the first ultrasonic TD array 31 and the second ultrasonic TD array 32.

The first ultrasonic TD array 31 and the second ultrasonic TD array 32 are separated from each other by a predetermined inter-probe distance Lp and are fixed to a sticky pedestal 34 in a state in which a scanning surface is parallel, and is configured to measure a short axis direction section of the blood vessel 5. Therefore, ultrasonic images including the short axis direction section of the blood vessel 5 are obtained at two locations through transmission and reception of ultrasonic waves performed by the first ultrasonic TD array 31 and transmission and reception performed by the second ultrasonic TD array 32. Since the first drive frequency for the first ultrasonic TD array 31 and the second drive frequency for the second ultrasonic TD array 32 are different from each other, ultrasonic waves can be transmitted and received in parallel, and thus ultrasonic images including the short axis direction section of the blood vessel 5 can be obtained simultaneously or nearly simultaneously. The inter-probe distance Lp is 1 cm or more and 10 cm or less.

The sticky pedestal 34 has a sticky layer which is attachable to and detachable from a skin surface, and thus is not easily peeled off even if the subject 3 moves the body thereof. The sticky pedestal 34 is attached so that the first ultrasonic TD array 31 and the second ultrasonic TD array 32 can draw the short axis of the blood vessel 5 (brachial artery in the present embodiment), and the first ultrasonic TD array 31 is located on a heart side (upstream side), and the second ultrasonic TD array 32 is located on a fingertip side (downstream side).

There may be a configuration in which the first ultrasonic TD array 31 and the second ultrasonic TD array 32 are respectively mounted on separate sticky pedestals 34 instead of being mounted on a single sticky pedestal 34.

The blood vessel 5 as a measurement target is not limited to brachial artery, and, for example, other artery such as radial artery or femoral artery may be a measurement target.

FIG. 3 is a schematic sectional view at positions where the first ultrasonic TD array 31 and the second ultrasonic TD array 32 are attached. The first ultrasonic TD array 31 and the second ultrasonic TD array 32 transmit ultrasonic waves corresponding to the first drive frequency and the second drive frequency to the blood vessel 5, and receive reflected waves from a front wall 5f and a rear wall 5r of the blood vessel 5, respectively. The control device 10 calculates a diameter of the blood vessel 5, that is, a first blood vessel diameter D1 measured with the first ultrasonic TD array 31 and a second blood vessel diameter D2 measured with the second ultrasonic TD array 32, on the basis of an arrival time difference between a received wave from the front wall 5f and a received wave from the rear wall 5r. Transmission of ultrasonic waves and reception of reflected waves are continuously performed in parallel to each other at a considerably short time interval by the first ultrasonic TD array 31 and the second ultrasonic TD array 32. Thus, the first blood vessel diameter D1 and the second blood vessel diameter D2 can also be continuously calculated in parallel to each other. As a result, a waveform in which a blood vessel diameter changes in a time series is obtained.

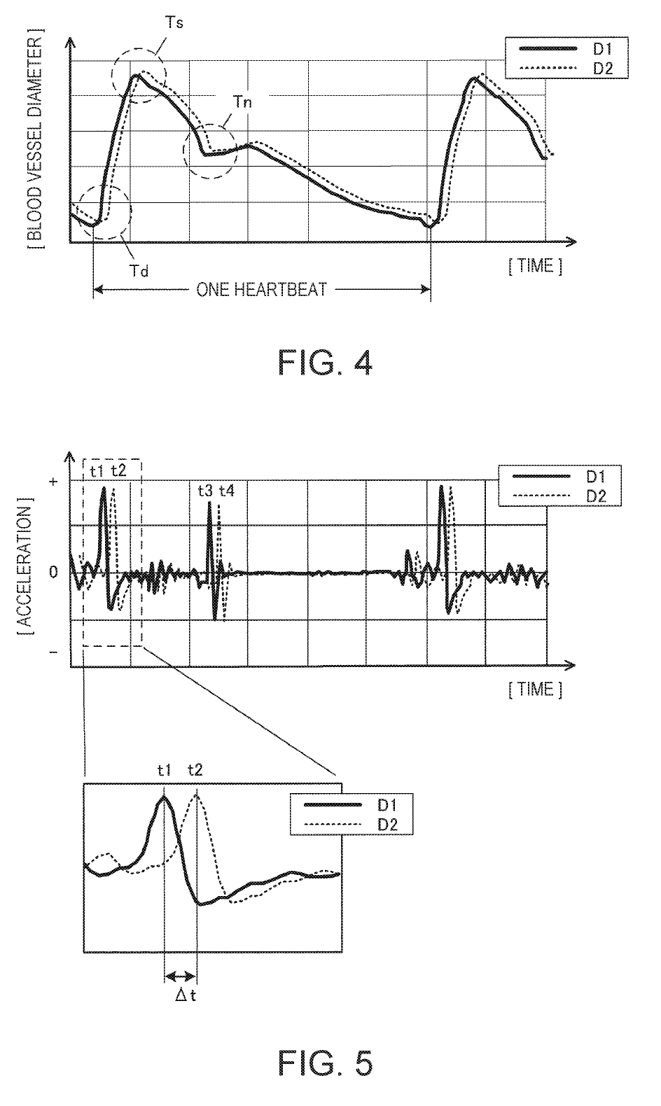

FIGS. 4 and 5 illustrate time-series waveform examples of the first blood vessel diameter D1 and the second blood vessel diameter D2. FIG. 4 illustrates blood vessel diameter waveforms, and FIG. 5 illustrates acceleration waveforms obtained by performing second order differentiation on the blood vessel diameters and also shows an enlarged view of the acceleration waveforms in a diastole. The waveforms are simplified for better understanding.

According to FIG. 4, a diastole Td, a systole Ts, and a notch timing Tn can be seen from changes in the first blood vessel diameter D1 and the second blood vessel diameter D2. Since the first ultrasonic TD array 31 is disposed further toward the heart side than the second ultrasonic TD array 32, a pressure wave caused by a systole arrives at the first ultrasonic TD array 31 earlier. Thus, extension and contraction timings of the first blood vessel diameter D1 are earlier than those of the second blood vessel diameter D2.

However, it cannot be said that actual changes in a blood vessel diameter clearly show the diastole Td, the systole Ts, and the notch timing Tn as illustrated in FIG. 4. Particularly, there are relatively many cases where a time point of a peak of the systole Ts cannot be clearly specified, and, for example, the influence of heart noise appears in the subject 3 whose heart disease is concerned.

Therefore, in the present embodiment, peaks at the diastole Td and the notch timing Tn are detected instead of a peak at the systole Ts. Specifically, the first blood vessel diameter D1 and the second blood vessel diameter D2 are subjected to second order differentiation one by one at a time point t, and thus accelerations of respective diameter changes are obtained. Peaks in which second order differential values satisfy a predetermined peak condition (for example, exceeding a reference value) are found, and thus the diastole Td and the notch timing Tn are detected. According to this method, the diastole Td and the notch timing Tn can be reliably found. The second order differentiation is an example of predetermined differentiation calculation.

The use of the second order differential values secondarily increases robustness in blood vessel diameter measurement. In other words, in a case where a direction (hereinafter, referred to as a “transmission line”) of an ultrasonic wave transmitted from the first ultrasonic TD array 31 or the second ultrasonic TD array 32 passes through the center of the short axis direction section of the blood vessel 5, the greatest fluctuation of a blood vessel diameter appears on the transmission line, and thus a change in the blood vessel diameter clearly appears in a waveform. However, if the transmission line is deviated relative to the center of the short axis direction section, a fluctuation in a blood vessel diameter is small, and thus a waveform is corrupted. In a configuration in which the diastole Td and the notch timing Tn are found on the basis of peaks of blood vessel diameter waveforms without performing differentiation calculation, if the subject 3 moves the body thereof, the transmission line is deviated relative to the blood vessel 5, peaks of blood vessel diameter waveforms are hardly expressed, and thus there is a probability that the diastole Td and the notch timing Tn may not be found, and therefore continuous measurement may be stopped. However, if second order differentiation is performed as in the present embodiment, clear peaks appear in acceleration waveforms as long as the wall portion of the blood vessel 5 is specified even in a state in which the transmission line does not pass through the center of the short axis direction section of the blood vessel 5. In other words, high robustness against body motion of the subject 3 is obtained. It is possible to reduce a probability that continuous measurement of vasculature biological information may be stopped, even if the subject 3 moves the body thereof.

The second order differentiation is described to be performed as differentiation calculation, but the diastole Td and the notch timing Tn may be detected by performing differentiation once. Even if differentiation is performed once, robustness against body motion of the subject 3 is high to some extent.

Meanwhile, a pulse wave propagation time difference Δt is obtained from peak time points t1 and t2 of second order differential values for the first blood vessel diameter D1 and the second blood vessel diameter D2. If the pulse wave propagation time difference Δt is obtained, pulse wave propagation velocity PWV can be obtained on the basis of the pulse wave propagation time difference Δt and the inter-probe distance Lp as a propagation length. Calculation of the pulse wave propagation velocity PWV is also performed on a difference between peak time points t3 and t4. The peak time points t1 and t2 are time points (timings) corresponding to the diastole Td, and the peak time points t3 and t4 are time points corresponding to the notch timing Tn. A diastole pulse wave propagation velocity PWVd and a notch-timing pulse wave propagation velocity PWVn which are pulse wave propagation velocities PWV at the diastole Td and the notch timing Tn are calculated, and an average thereof is used as a final pulse wave propagation velocity PWVa.

In the present embodiment, the diastole pulse wave propagation velocity PWVd and the notch-timing pulse wave propagation velocity PWVn are calculated, and the average pulse wave propagation velocity PWVa is used as a final pulse wave propagation velocity, but either one of the diastole pulse wave propagation velocity PWVd and the notch-timing pulse wave propagation velocity PWVn may be calculated, and a calculated value may be used as a final pulse wave propagation velocity.

Interface between Ultrasonic Probe and Control Device

Meanwhile, the drive frequencies for the first ultrasonic TD array 31 and the second ultrasonic TD array 32 are different from each other, but the ultrasonic probe 30 is connected to the control device 10 via the common signal line CL as illustrated in FIG. 1. In other words, the first ultrasonic TD array 31 and the second ultrasonic TD array 32 are connected in common to the common signal line CL which is used for both transmission and reception.

Thus, there is a need of technical examination for driving the first ultrasonic TD array 31 and the second ultrasonic TD array 32 in order to transmit ultrasonic waves from the first ultrasonic TD array 31 and the second ultrasonic TD array 32.

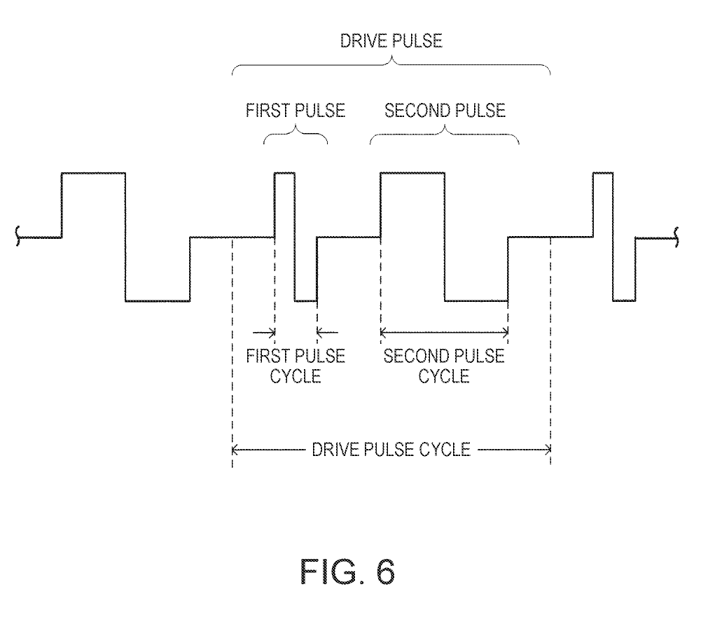

FIG. 6 is a diagram illustrating an example of a waveform of a drive signal for transmitting ultrasonic waves from the first ultrasonic TD array 31 and the second ultrasonic TD array 32. The drive signal is a signal in which a drive pulse is repeated in a drive pulse cycle, and the drive pulse includes a first pulse and a second pulse in a time division manner.

The first pulse is a pulse wave in a first pulse cycle corresponding to the first drive frequency, and the second pulse is a pulse wave in a second pulse cycle corresponding to the second drive frequency. In other words, the first ultrasonic TD array 31 transmits ultrasonic waves in response to the first pulse, and does not respond to the second pulse. This is because the first ultrasonic TD array 31 is driven at the first drive frequency. On the contrary, the second ultrasonic TD array 32 transmits ultrasonic waves in response to the second pulse, and does not respond to the first pulse. This is because the second ultrasonic TD array 32 is driven at the second drive frequency.

Consequently, the first ultrasonic TD array 31 and the second ultrasonic TD array 32 respectively transmit ultrasonic waves at corresponding drive frequencies by using a single drive signal supplied via the common signal line CL.

In a case where reflected waves of the ultrasonic waves are received, the first ultrasonic TD array 31 and the second ultrasonic TD array 32 respond to reflected waves corresponding to the drive frequencies, and output signals of the received waves (received signals) corresponding to the drive frequencies to the common signal line CL. In other words, a mixed received signal in which individual received signals of the first ultrasonic TD array 31 and the second ultrasonic TD array 32 are mixed with each other is carried through the common signal line CL. However, frequencies of the respective individual received signals are the first drive frequency and the second drive frequency which are different from each other, and can thus be separated and extracted by the control device 10 by using a filter circuit.

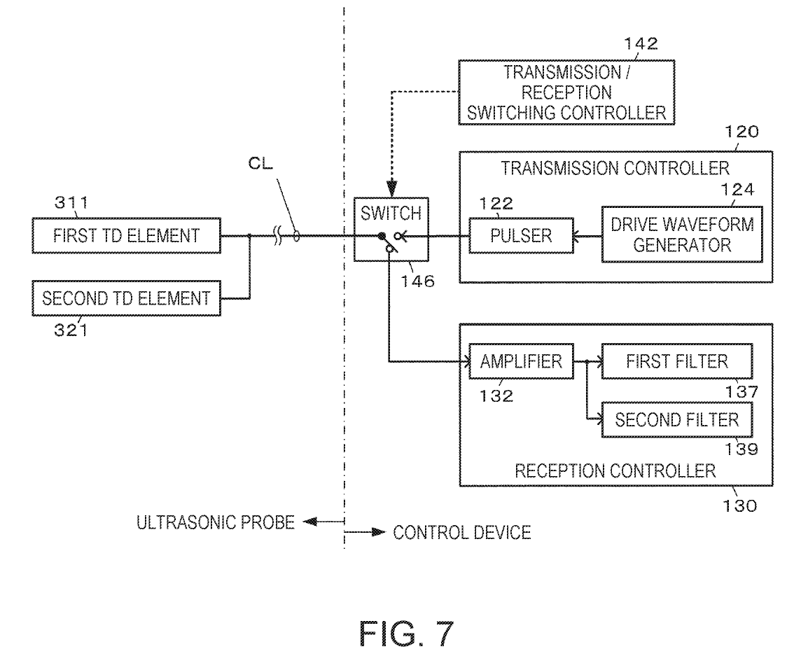

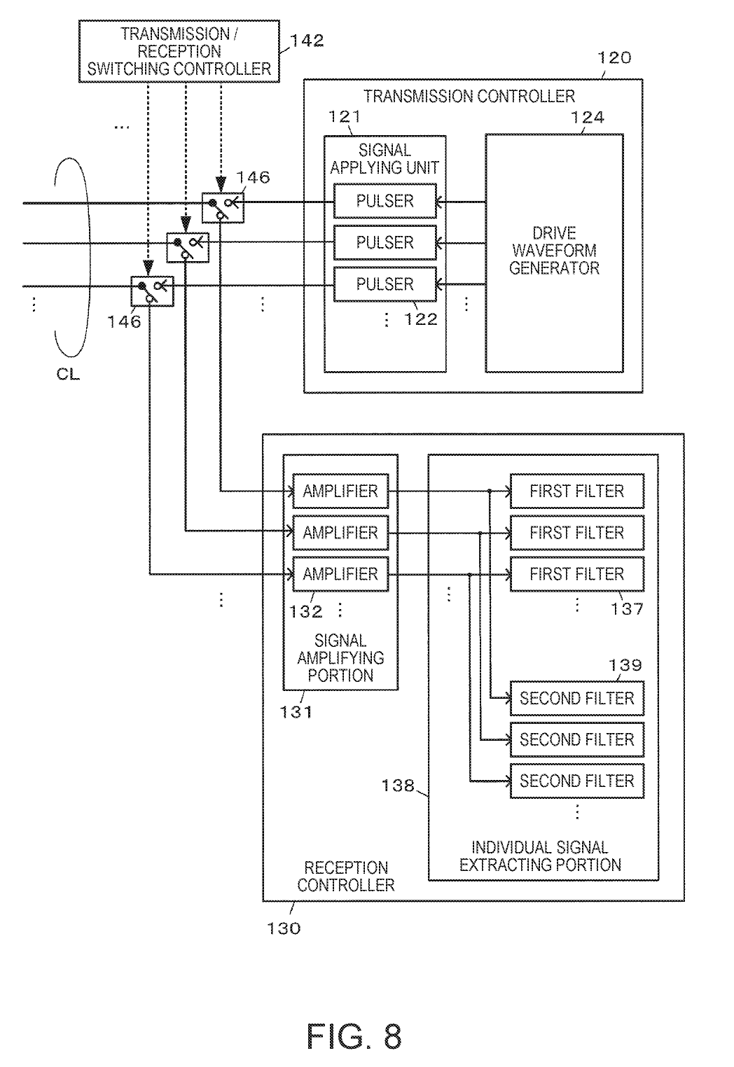

With reference to FIGS. 7 and 8, a configuration of the control device 10 related to transmission and reception of ultrasonic waves will be described more in detail. FIG. 7 is a diagram illustrating a configuration related to transmission and reception in a case of focusing on a first TD element 311 which is a single ultrasonic transducer element (hereinafter, abbreviated a “TD element”) forming the first ultrasonic TD array 31, and a second TD element 321 which is a single TD element forming the second ultrasonic TD array 32. FIG. 8 is a diagram illustrating a configuration in which the configuration focusing on the single TD element illustrated in FIG. 7 is viewed as a whole of the ultrasonic probe 30.

Focusing on FIG. 7, the control device 10 includes a transmission controller 120, a reception controller 130, a transmission/reception switching controller 142, and a switch 146. The transmission controller 120 is a functional unit which performs control for transmitting ultrasonic waves from the first TD element 311 and the second TD element 321, and is provided with a pulser 122 and a drive waveform generator 124. The drive waveform generator 124 generates a waveform of the drive pulse including the first pulse and the second pulse illustrated in FIG. 6 in a time division manner, and instructs the pulser 122 to repeatedly output the generated waveform in a drive pulse cycle. The pulser 122 generates a voltage pulse according to the waveform for which the instruction is given by the drive waveform generator 124, and applies the voltage pulse to the common signal line CL as a drive signal via the switch 146. The common signal line CL is connected in common to the first TD element 311 and the second TD element 321.

Here, the switch 146 is a switch circuit which switches connection of the common signal line CL between the transmission controller 120 and the reception controller 130. The transmission/reception switching controller 142 causes the switch 146 to perform switching in accordance with transmission and reception timings of ultrasonic waves.

If the drive signal is transmitted to the first TD element 311 and the second TD element 321 via the common signal line CL, the first TD element 311 transmits ultrasonic waves corresponding to the first drive frequency, and the second TD element 321 transmits ultrasonic waves corresponding to the second drive frequency. The transmissions of the ultrasonic waves are performed in parallel.

Next, in reception of reflected waves of the ultrasonic waves, an individual received signal received by the first TD element 311 and an individual received signal received by the second TD element 321 are mixed with each other since the first TD element 311 and the second TD element 321 are connected in common to the common signal line CL, and a mixed received signal is carried through the common signal line CL.

During reception of the ultrasonic waves, the switch 146 performs switching so that the common signal line CL is connected to the reception controller 130 under the control of the transmission/reception switching controller 142. Thus, the mixed received signal is input to the reception controller 130.

The reception controller 130 includes an amplifier 132, a first filter 137, and a second filter 139. The amplifier 132 is a circuit which amplifies a level of the mixed received signal. The mixed received signal which has been amplified is input to the first filter 137 and the second filter 139. The first filter 137 is a frequency filter through which a band of the first drive frequency passes and which cuts off other bands, and the second filter 139 is a frequency filter through which a band of the second drive frequency passes and which cuts off other bands. Therefore, the individual received signal received by the first TD element 311 is selected and extracted by the first filter 137, and the individual received signal received by the second TD element 321 is selected and extracted by the second filter 139.

The first ultrasonic TD array 31 includes a plurality of first TD elements 311, and the second ultrasonic TD array 32 includes a plurality of second TD elements 321. Thus, the functional block illustrated in FIG. 7 is provided in the control device 10 for each pair (combination) of the first TD element 311 and the second TD element 321.

More specifically, as illustrated in FIG. 8, the transmission controller 120 includes a plurality of pulsers 122 corresponding to pairs (combinations) of the first TD elements 311 and the second TD elements 321. The pulsers 122 are a functional unit which applies a drive signal to the common signal line CL, and may thus be referred to as a signal applying unit 121.

The reception controller 130 includes a plurality of amplifiers 132, a plurality of first filters 137, and a plurality of second filters 139, corresponding to pairs (combinations) of the first TD elements 311 and the second TD elements 321. The plurality of amplifiers 132 may be collectively referred to as a signal amplifying portion 131, and the plurality of first filters 137 and the plurality of second filters 139 may be referred to as an individual signal extracting portion 138.

### Description of Functional Configuration of Measurement Apparatus 1

Next, a description will be made of a functional configuration of the entire measurement apparatus 1 for realizing the present embodiment.

FIG. 9 is a block diagram illustrating a functional configuration example of the measurement apparatus 1 of the present embodiment. The measurement apparatus 1 includes the control device 10 and the ultrasonic probe 30. The control device 10 includes the processing unit 100, the operation input unit 200, the display unit 400, the sound output unit 430, the communication unit 450, and the storage unit 500.

The ultrasonic probe 30 includes the first ultrasonic TD array 31 and the second ultrasonic TD array 32. The first ultrasonic TD array 31 and the second ultrasonic TD array 32 transmit ultrasonic waves for ultrasonic measurement on the basis of a drive signal which is transmitted from the processing unit 100 via the common signal line CL, receive reflected waves thereof, and transmits received signals to the processing unit 100 via the common signal line CL.

The processing unit 100 generally controls the measurement apparatus 1, and performs various calculation processes related to measurement of vasculature biological information of the subject 3. The processing unit 100 is implemented by, for example, a microprocessor such as a central processing unit (CPU) or a graphics processing unit (GPU), or an electronic component such as an application specific integrated circuit (ASIC), a field programmable gate array (FPGA), or an integrated circuit (IC) memory. The processing unit 100 performs input/output control of data between the respective functional units, performs various calculation processes on the basis of a predetermined program or data, or an operation input signal from the operation input unit 200, and calculates vasculature biological information (pulse wave propagation velocity in the present embodiment) of the subject 3.

The processing unit 100 includes an ultrasonic wave transmission/reception controller 110, a vasculature biological information measurement section 150, a display information generation section 170, and a clocking section 180.

The ultrasonic wave transmission/reception controller 110 controls transmission and reception of ultrasonic waves in the ultrasonic probe 30. A specific configuration thereof is the same as described with reference to FIGS. 7 and 8. An individual received signal received by the first ultrasonic TD array 31 and an individual received signal received by the second ultrasonic TD array 32 are stored in the storage unit 500 as received data 514 in a time series in correlation with measurement time points.

The vasculature biological information measurement section 150 is a functional section which measures vasculature biological information on the basis of the received data 514 which is obtained through transmission and reception of ultrasonic waves under the control of the ultrasonic wave transmission/reception controller 110. The vasculature biological information measurement section 150 includes a blood vessel diameter measurement portion 152, a feature timing determination portion 154, a heartbeat determination portion 156, a blood vessel position determination portion 158, a propagation length setting portion 162, and a pulse wave propagation velocity calculation portion 164.

The blood vessel diameter measurement portion 152 continuously measures a blood vessel diameter of the blood vessel 5 (brachial artery in the present embodiment) on the basis of the received data 514. A temporal change waveform of the blood vessel diameter is obtained through this continuous measurement. In the present embodiment, the first blood vessel diameter D1 is measured by using data received by the first ultrasonic TD array 31, and the second blood vessel diameter D2 is measured by using data received by the second ultrasonic TD array 32. When the blood vessel diameter is measured, the front wall 5f and the rear wall 5r of the blood vessel 5 are detected from received signals (refer to FIG. 3), and a distance difference between the front wall 5f and the rear wall 5r is obtained, but the blood vessel diameter may be measured according to other methods.

The feature timing determination portion 154 determines a first timing and a second timing on the basis of the temporal change waveform of the blood vessel diameter measured by the blood vessel diameter measurement portion 152. In the present embodiment, the first timing is the diastole Td, and the second timing is the notch timing Tn, but, of course, the reverse thereof may occur. The feature timing determination portion 154 determines the diastole Td and the notch timing Tn corresponding to the first blood vessel diameter D1, and the diastole Td and the notch timing Tn corresponding to the second blood vessel diameter D2. The blood vessel diameter measurement portion 152 specifies a blood vessel diameter in the feature timing determined by the feature timing determination portion 154. Specifically, in the changing first blood vessel diameter D1, a diastole blood vessel diameter Dd corresponding to the diastole Td, and a notch-timing blood vessel diameter Dn corresponding to the notch timing Tn are specified. In addition, in the changing second blood vessel diameter D2, a diastole blood vessel diameter Dd corresponding to the diastole Td, and a notch-timing blood vessel diameter Dn corresponding to the notch timing Tn are specified.

The feature timing determination portion 154 performs predetermined differentiation calculation on the temporal change waveform of the blood vessel diameter so as to determine the diastole Td and the notch timing Tn which are feature timings of pulse waves. In the present embodiment, second order differentiation is performed, a time point (timing) satisfying a peak condition indicating that a second order differential value is equal to or greater than a reference value is detected, and thus a feature timing is determined.

The heartbeat determination portion 156 determines a heartbeat break in the ultrasonic measurement on the basis of the determination result in the feature timing determination portion 154. The heartbeat determination portion 156 may have a function of calculation a heart rate.

The blood vessel position determination portion 158 determines a relative position (referred to as a “relative blood vessel position”) of the blood vessel 5 as a measurement target with the first ultrasonic TD array 31 as a reference on the basis of the data received by the first ultrasonic TD array 31, and relative blood vessel position of the blood vessel 5 as a measurement target with the second ultrasonic TD array 32 as a reference on the basis of the data received by the second ultrasonic TD array 32. The determined relative blood vessel positions are stored in the storage unit 500 as relative blood vessel position data 516 in a time series in correlation with measurement time points.

The propagation length setting portion 162 sets a distance between a measurement position of the first ultrasonic TD array 31 and a measurement position of the second ultrasonic TD array 32 in the blood vessel 5 as a measurement target, as a propagation length over which a pulse wave propagates, on the basis of the inter-probe distance Lp and the relative blood vessel position data 516. In a case where the relative blood vessel position for the first ultrasonic TD array 31 and the relative blood vessel position for the second ultrasonic TD array 32 satisfy a predetermined equal condition, the blood vessel 5 runs through the body of the subject 3 so as to be parallel in the same manner as in the relative positional relationship between the first ultrasonic TD array 31 and the second ultrasonic TD array 32. Thus, the inter-probe distance Lp may be set as a propagation length as it is. In a case where the relative blood vessel position for the first ultrasonic TD array 31 and the relative blood vessel position for the second ultrasonic TD array 32 do not satisfy the equal condition, the inter-probe distance Lp is corrected according to a difference therebetween, so as to be set as a propagation length. Specifically, a correction value is calculated so as to be proportional to a distance between plots on the basis of the distance between plots when the relative blood vessel position for the first ultrasonic TD array 31 and the relative blood vessel position for the second ultrasonic TD array 32 are plotted on the same plane, and a propagation length is set by adding the correction value to the inter-probe distance Lp.

Either or both of the relative blood vessel position for the first ultrasonic TD array 31 and the relative blood vessel position for the second ultrasonic TD array 32 may change due to body motion of the subject 3, but, according to the present embodiment, a propagation length can be calculated and set so as to track the body motion.

The propagation length set by the propagation length setting portion 162 is stored in the storage unit 500 as propagation length data 540. Setting of the propagation length is repeatedly performed for each measurement time point, and thus set propagation lengths are stored in the propagation length data 540 in a time series in correlation with measurement time points.

The pulse wave propagation velocity calculation portion 164 measures the pulse wave propagation velocity PWV in the blood vessel 5. In the present embodiment, a pulse wave propagation time difference Δt at each feature timing of the diastole Td and the notch timing Tn is calculated, and the pulse wave propagation velocity PWV is on the basis of on the basis of the time difference Δt and the propagation length stored in the propagation length data 540. In other words, the diastole pulse wave propagation velocity PWVd and the notch-timing pulse wave propagation velocity PWVn are obtained. The average pulse wave propagation velocity PWVa obtained by averaging the diastole pulse wave propagation velocity PWVd and the notch-timing pulse wave propagation velocity PWVn is used as a final pulse wave propagation velocity. Either one of the diastole pulse wave propagation velocity PWVd and the notch-timing pulse wave propagation velocity PWVn may be calculated, and a calculated value may be used as a final pulse wave propagation velocity.

The display information generation section 170 generates various operation screens required in measurement of vasculature biological information or images for displaying a measurement result, and outputs the screens or the images to the display unit 400. The display unit 400 is a device displaying image data from the display information generation section 170.

The clocking section 180 measures a measurement time point. A clocking method may be selected as appropriate, and, for example, a system clock may be used.

The operation input unit 200 receives a user's various input operations, and outputs an input operation signal corresponding to an input operation to the processing unit 100. The operation input unit 200 is formed of a button switch, a lever switch, a dial switch, a track pad, a mouse, a touch panel, or the like.

The sound output unit 430 is a device which outputs a sound based on audio signal output from the processing unit 100, and is, for example, a speaker. The sound output unit 430 preferably generates a notification sound, for example, in a case where blood pressure reaches a predetermined abnormal value.

The communication unit 450 is a communication device performing communication with an external apparatus of the measurement apparatus 1. The communication may be wired communication or wireless communication. The communication unit 450 may transmit measured data to the external apparatus.

The storage unit 500 is formed of a recording medium such as an IC memory, a hard disk, or an optical disc, and stores various programs, or various data such as data during calculation in the processing unit 100. The storage unit 500 of the control device 10 may be configured to be provided as a separate device and to be communicably connected to the processing unit 100 via a communication line such as a local area network (LAN) or the Internet. For example, in this case, the storage unit 500 may be implemented as a storage device of a server connected to the Internet.

The storage unit 500 stores a measurement program 510, the received data 514, the relative blood vessel position data 516, blood vessel diameter log data 520, pulse wave propagation velocity log data 530, and the propagation length data 540. Of course, in addition thereto, various determination flags, clocking counter values, and the like may be stored as appropriate.

The measurement program 510 is executed by the processing unit 100, so as to realize functions such as the ultrasonic wave transmission/reception controller 110, the vasculature biological information measurement section 150, the display information generation section 170, and the clocking section 180. Any one of the functional sections may be implemented by hardware such as an electronic circuit.

The measurement program 510 includes, as a sub-routine program, an ultrasonic wave transmission/reception control program 512 for controlling transmission and reception of ultrasonic waves for the ultrasonic probe 30 so as to realize a function of the ultrasonic wave transmission/reception controller 110.

The received data 514 is data in which an individual received signal in the first ultrasonic TD array 31 and an individual received signal in the second ultrasonic TD array 32, obtained through transmission of ultrasonic waves and reception of reflected waves continuously performed by the ultrasonic probe 30 at a very short time interval from starting of measurement to ending thereof under the control of the ultrasonic wave transmission/reception controller 110, are stored in a time series in correlation with measurement time points.

The relative blood vessel position data 516 is data in which data regarding the relative blood vessel position for the first ultrasonic TD array 31 and data regarding the relative blood vessel position for the second ultrasonic TD array 32, determined by the blood vessel position determination portion 158, are stored in a time series in correlation with measurement time points.

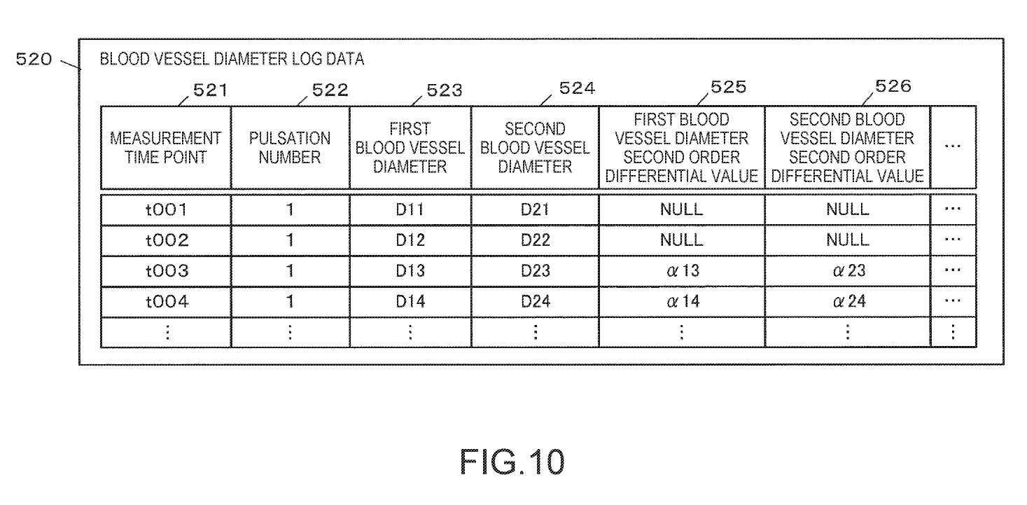

The blood vessel diameter log data 520 is data in which various data regarding a blood vessel diameter of the blood vessel 5 is stored in correlation with measurement time points. Specifically, as illustrated in FIG. 10, the blood vessel diameter log data 520 stores, in correlation with a measurement time point 521 for each measurement cycle, a pulsation number 522 (for example, a value indicating what number of pulsation from starting of measurement) for identifying a pulsation at the time point, a first blood vessel diameter 523 and a second blood vessel diameter 524 measured at that time, a first blood vessel diameter second order differential value 525, and a second blood vessel diameter second order differential value 526. Of course, other data may be stored as appropriate. The measurement time point 521 gradually elapses as “t001”, “t002”, “t003”, and “t004”, but the pulsation number 522 is “1” in all cases, and thus FIG. 10 illustrates that the data is data regarding the same pulsation. The first blood vessel diameter 523 and the second blood vessel diameter 524 are acquired in a time series, and thus temporal change waveforms of the blood vessel diameters are obtained. The reason why the first blood vessel diameter second order differential value 525 and the second blood vessel diameter second order differential value 526 are “NULL” at the time points “t001” and “t002” is that there is no data before the time point “t001”, and thus data required to calculate a second order differential value cannot be obtained.

The pulsation number 522 is stored on the basis of determination of a heartbeat in the heartbeat determination portion 156, and measurement results in the blood vessel diameter measurement portion 152 are stored as the first blood vessel diameter 523 and the second blood vessel diameter 524. The first blood vessel diameter second order differential value 525 and the second blood vessel diameter second order differential value 526 are values calculated when the feature timing determination portion 154 determines featuring timings.

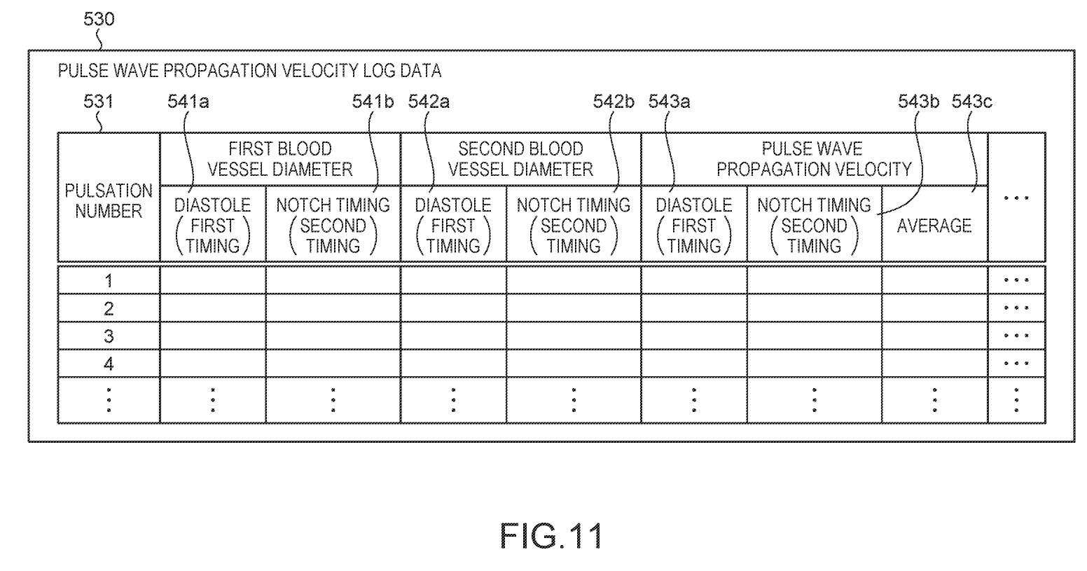

The pulse wave propagation velocity log data 530 stores pulse wave propagation velocity and data used to calculate the pulse wave propagation velocity. Specifically, as illustrated in FIG. 11, the pulse wave propagation velocity log data 530 stores, in correlation with a pulsation number 531 corresponding to the pulsation number 522 of the blood vessel diameter log data 520, a diastole blood vessel diameter 541a and a notch-timing blood vessel diameter 541b related to the first blood vessel diameter D1, a diastole blood vessel diameter 542a and a notch-timing blood vessel diameter 542b related to the second blood vessel diameter D2, and a diastole pulse wave propagation velocity 543a, a notch-timing pulse wave propagation velocity 543b, and an average pulse wave propagation velocity 543c, in the pulsation.

Blood vessel diameters at the feature timings performed by the blood vessel diameter measurement portion 152 are stored as the diastole blood vessel diameter 541a and the notch-timing blood vessel diameter 541b related to the first blood vessel diameter D1, and the diastole blood vessel diameter 542a and the notch-timing blood vessel diameter 542b related to the second blood vessel diameter D2. Pulse wave propagation velocities calculated by the pulse wave propagation velocity calculation portion 164 are stored as the diastole pulse wave propagation velocity 543a, the notch-timing pulse wave propagation velocity 543b, and the average pulse wave propagation velocity 543c.

Propagation lengths calculated by the propagation length setting portion 162 are stored as the propagation length data 540 in a time series in correlation with measurement time points.

### Description of Flow of Processes

Next, an operation of the measurement apparatus 1 will be described.

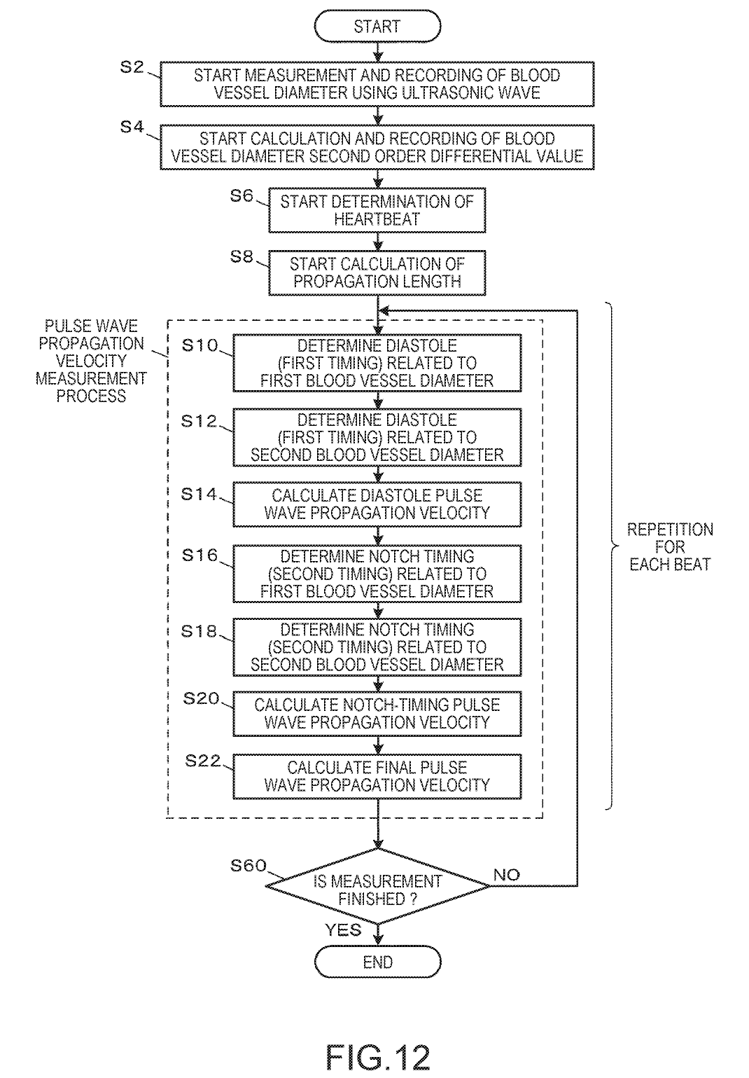

FIG. 12 is a flowchart for explaining a flow of principal processes in the measurement apparatus 1 according to the present embodiment, and illustrates a flow of processes performed by executing the measurement program 510. It is assumed that the ultrasonic probe 30 is attached at a predetermined position of the subject 3 in advance.

First, the processing unit 100 starts ultrasonic measurement using the ultrasonic probe 30, and starts measurement and recording of a blood vessel diameter D (step S2). The processing unit 100 starts calculation and recording of a second order differential value of the blood vessel diameter D (step S4). Data is stored in the blood vessel diameter log data 520 through steps S2 and S4.

The processing unit 100 determines a heartbeat break on the basis of a change in the blood vessel diameter D so as to start a heartbeat (step S6). Identification information regarding the determined heartbeat is stored in the pulsation number 522 of the blood vessel diameter log data 520 as a heartbeat number from the starting of the measurement. The processing unit 100 starts calculation and setting of a propagation length (step S8), and stores the propagation length in the propagation length data 540.

Next, the processing unit 100 performs a pulse wave propagation velocity measurement process (steps S10 to S22).

Specifically, the processing unit 100 determines a diastole related to the first blood vessel diameter D1 and a diastole related to the second blood vessel diameter D2 on the basis of the blood vessel diameter log data 520 (steps S10 to S12). Blood vessel diameters at the diastoles are specified. A time difference between the two diastoles, that is, a diastole pulse wave propagation time difference Δtd is obtained, and a diastole pulse wave propagation velocity PWVd is calculated on the basis of the diastole pulse wave propagation time difference Δtd and a propagation length at the diastole read from the propagation length data 540 (step S14).

Successively, the processing unit 100 determines a notch timing related to the first blood vessel diameter D1 and a notch timing related to the second blood vessel diameter D2 on the basis of the blood vessel diameter log data 520 (steps S16 to S18). Blood vessel diameters at the notch timings are specified. A time difference between the two notch timings, that is, a notch-timing pulse wave propagation time difference Δtn is obtained, and a notch-timing pulse wave propagation velocity PWVn is calculated on the basis of the notch-timing pulse wave propagation time difference Δtn and a propagation length at the notch timing read from the propagation length data 540 (step S20).

The diastole pulse wave propagation velocity PWVd and the notch-timing pulse wave propagation velocity PWVn at the same beat are averaged, and thus an average pulse wave propagation velocity PWVa is calculated so as to be used as a final pulse wave propagation velocity (step S22). Each data item calculated in the pulse wave propagation velocity measurement process is stored in the pulse wave propagation velocity log data 530.

The processing unit 100 repeatedly performs the process in steps S10 to S22 for each heartbeat until it is determined that measurement is finished, for example, as a result of an input operation for finishing the measurement (NO in step S60).

As mentioned above, according to the present embodiment, the ultrasonic probe 30 includes the first ultrasonic TD array 31 and the second ultrasonic TD array 32 for which drive frequencies are different from each other. Thus, it is possible to reduce the occurrence of interference in transmission and reception of ultrasonic waves. Therefore, it is possible to perform parallel transmission and reception of ultrasonic waves at two locations. The first ultrasonic TD array 31 and the second ultrasonic TD array 32 are connected in common to the common signal line CL which is used for both transmission and reception. Consequently, it is possible to reduce the number of signal cables.

An applicable form of the invention is not limited to the above-described embodiment, and a constituent element may be added, omitted, or changed as appropriate.

For example, vasculature biological information measured by the above-described measurement apparatus 1 is not limited to a blood vessel diameter or pulse wave propagation velocity, and a blood flow rate or blood pressure may be measured. The blood flow rate may be obtained by using Doppler frequencies of transmitted and received ultrasonic waves. The blood pressure may be estimated and calculated from a blood vessel system by using a blood vessel elasticity index value (for example, stiffness parameter β).

The ultrasonic probe 30 may be configured to include ultrasonic TD arrays of N types (where N≧2) of two or more types for which drive frequencies are different from each other. Also in this case, the ultrasonic TD arrays of N types may be configured to be connected in common to the common signal line CL which is used for both transmission and reception.

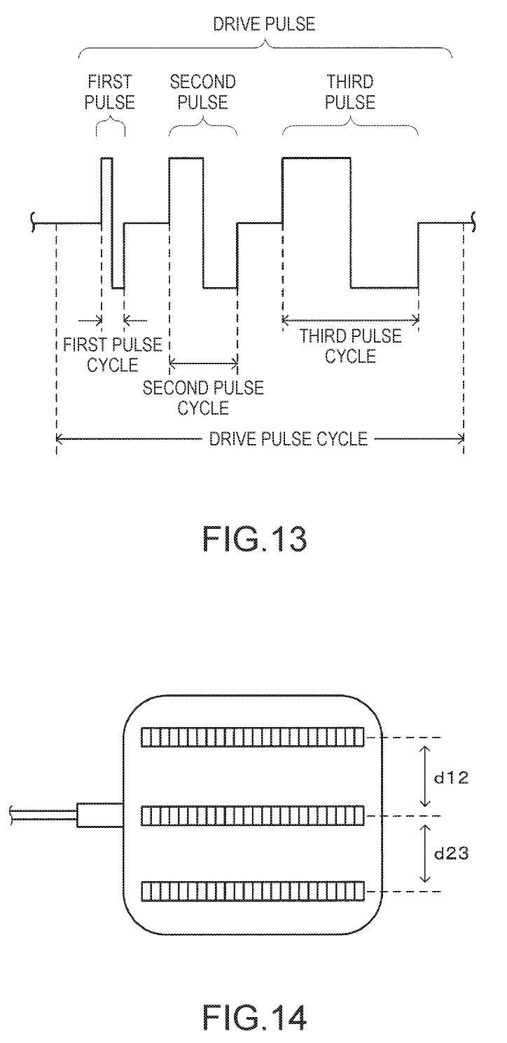

More specifically, in a case of N=3, as illustrated in FIG. 13, a drive pulse including a first pulse for a first ultrasonic TD array, a second pulse for a second ultrasonic TD array, and a third pulse for a third ultrasonic TD array in a time division manner may be applied to the common signal line CL by the transmission controller 120, so that the first to third ultrasonic TD arrays are driving in parallel, and thus ultrasonic waves may be transmitted in parallel.

The first to third ultrasonic TD arrays in this case may be configured to be disposed as illustrated in FIG. 14, for example. In other words, the ultrasonic TD arrays of N types (three types in this case) are provided to be disposed in parallel and in a column. With this arrangement configuration, it is possible to implement a configuration suitable for performing ultrasonic measurement on the same blood vessel 5 as a measurement target at different positions.

In a case where ultrasonic TD arrays of N types (where N≧2) are disposed in parallel and in a column, a gap between adjacent ultrasonic TD arrays is preferably 1 cm or more and 10 cm or less since vasculature biological information is measured for the same blood vessel 5. In the example illustrated in FIG. 14, each of gaps d12 and d23 is 1 cm or more and 10 cm or less.

In a case where the ultrasonic probe 30 is configured to include ultrasonic TD arrays of N types (where N≧2), a drive frequency of each ultrasonic TD array is set so that, in the order starting from the highest drive frequency for the ultrasonic TD arrays, an immediately higher drive frequency is twice or more higher than an immediately lower drive frequency. In the above-described manner, it is possible to effectively prevent interference between transmitted or received ultrasonic waves.

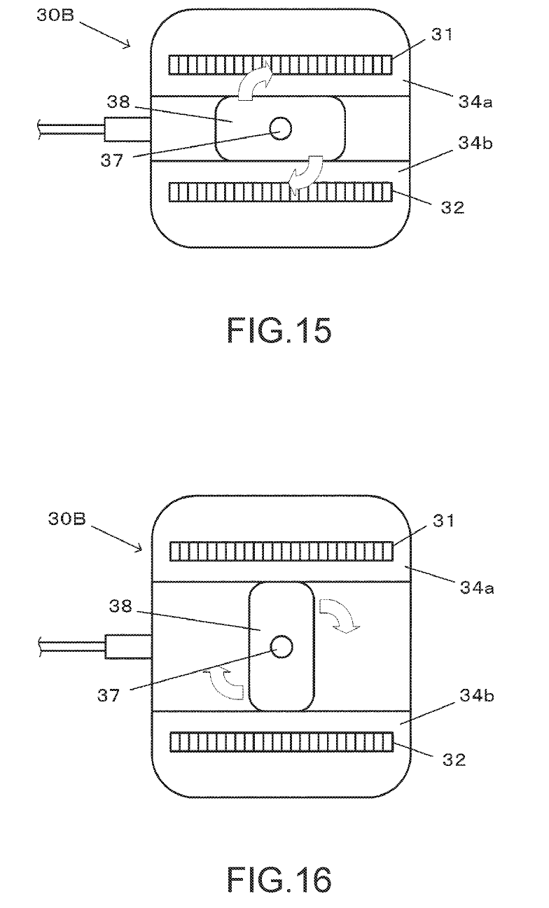

In the above-described embodiment, a gap between the ultrasonic TD arrays is set to be constant, but, there may be a configuration changing the gap is incorporated into the ultrasonic probe 30, so as to change the gap between the ultrasonic TD arrays. FIGS. 15 and 16 illustrate a configuration example of an ultrasonic probe 30B.

FIGS. 15 and 16 are diagrams illustrating a configuration example of the ultrasonic probe 30B changing a gap between the ultrasonic TD arrays, and is a schematic diagram of a rear surface (an attachment surface on which the ultrasonic probe is attached to the skin of the subject 3) side of the ultrasonic probe 30B.

In the ultrasonic probe 30B, the first ultrasonic TD array 31 is mounted on an upper half sticky pedestal member 34a in FIGS. 15 and 16, and the second ultrasonic TD array 32 is mounted on a lower half sticky pedestal member 34b in FIGS. 15 and 16. A rotation body 38 which rotates about a central axis 37 is provided to be parallel to the rear surface of the ultrasonic probe 30B between the half sticky pedestal member 34a and the half sticky pedestal member 34b. The half sticky pedestal member 34a and the half sticky pedestal member 34b are mounted to be slid in directions of coming close to and/or being separated from each other (vertical direction in FIGS. 15 and 16), and are biased in the direction of coming close to each other by an elastic member such as rubber. The rotation body 38 has a rectangular shape whose corners are chamfered in a plan view.

Therefore, if a longitudinal direction of the rotation body 38 is set to a horizontal direction as illustrated in FIG. 15, the first ultrasonic TD array 31 and the second ultrasonic TD array 32 are configured to be disposed with a first gap. On the other hand, if the rotation body 38 is rotated, and thus the longitudinal direction thereof is set to a vertical direction as illustrated in FIG. 16, the first ultrasonic TD array 31 and the second ultrasonic TD array 32 are configured to be disposed with a second gap. A difference the first gap and the second gap is a difference between the long side and the short side of the rotation body 38.

In a case where a gap between the ultrasonic TD arrays is changed, the propagation length setting portion 162 manually acquires a currently set gap (inter-probe distance Lp) through an input operation and sets a propagation length, or automatically acquires the currently set gap from the ultrasonic probe 30B and sets a propagation length.

The entire disclosure of Japanese Patent Application No. 2015-229830 filed on Nov. 25, 2015 is expressly incorporated by reference herein.

What is claimed is:

1. An ultrasonic probe comprising:
ultrasonic transducer arrays of N types (where N≧2) for which drive frequencies are different from each other; and
a common signal line that is used for both transmission and reception and to which the ultrasonic transducer arrays are connected in common.

2. The ultrasonic probe according to claim 1,
wherein the ultrasonic transducer arrays of N types are provided to be parallel to each other in a column.

3. The ultrasonic probe according to claim 2,
wherein a gap between the ultrasonic transducer arrays which are adjacent to each other is 1 cm or more and 10 cm or less.

4. The ultrasonic probe according to claim 2,
wherein a gap between the ultrasonic transducer arrays is configured to be changed.

5. The ultrasonic probe according to claim 1,
wherein, in the ultrasonic transducer arrays of N types, an immediately higher drive frequency is twice or more higher than an immediately lower drive frequency in the order starting from the highest drive frequency.

6. A control device controlling the ultrasonic probe according to claim 1, comprising:
a transmission controller that generates a drive signal for driving the ultrasonic transducer arrays of N types, and outputs the drive signal to the common signal line; and
a reception controller that receives a mixed received signal from the common signal line, and extracts an individual received signal for each of the ultrasonic transducer arrays.

7. The control device according to claim 6,
wherein the transmission controller generates the drive signal as a signal including drive waveforms corresponding to drive frequencies for the respective ultrasonic transducer arrays of N types in a time division manner.

8. The control device according to claim 6,
wherein the reception controller includes a filter unit corresponding to a drive frequency for each of the ultrasonic transducer arrays of N types.

9. A measurement apparatus comprising:
the ultrasonic probe according to claim 1 that performs transmission and reception of ultrasonic waves toward the same blood vessel of a subject in parallel; and
the control device according to claim 6,
wherein the measurement apparatus measures vasculature biological information.

10. A measurement apparatus comprising:
the ultrasonic probe according to claim 1 that performs transmission and reception of ultrasonic waves toward the same blood vessel of a subject in parallel; and
the control device according to claim 7,
wherein the measurement apparatus measures vasculature biological information.

11. A measurement apparatus comprising:
the ultrasonic probe according to claim 2 that performs transmission and reception of ultrasonic waves toward the same blood vessel of a subject in parallel; and
the control device according to claim 6,
wherein the measurement apparatus measures vasculature biological information.

12. A measurement apparatus comprising:
the ultrasonic probe according to claim 2 that performs transmission and reception of ultrasonic waves toward the same blood vessel of a subject in parallel; and
the control device according to claim 7,
wherein the measurement apparatus measures vasculature biological information.

13. A measurement apparatus comprising:
the ultrasonic probe according to claim 3 that performs transmission and reception of ultrasonic waves toward the same blood vessel of a subject in parallel; and
the control device according to claim 6,
wherein the measurement apparatus measures vasculature biological information.

14. A measurement apparatus comprising:
the ultrasonic probe according to claim 3 that performs transmission and reception of ultrasonic waves toward the same blood vessel of a subject in parallel; and
the control device according to claim 7,
wherein the measurement apparatus measures vasculature biological information.

15. A measurement apparatus comprising:
the ultrasonic probe according to claim 4 that performs transmission and reception of ultrasonic waves toward the same blood vessel of a subject in parallel; and
the control device according to claim 6,
wherein the measurement apparatus measures vasculature biological information.

16. A measurement apparatus comprising:
the ultrasonic probe according to claim 4 that performs transmission and reception of ultrasonic waves toward the same blood vessel of a subject in parallel; and
the control device according to claim 7,
wherein the measurement apparatus measures vasculature biological information.

17. A measurement apparatus comprising:
the ultrasonic probe according to claim 5 that performs transmission and reception of ultrasonic waves toward the same blood vessel of a subject in parallel; and
the control device according to claim 6,
wherein the measurement apparatus measures vasculature biological information.

18. A measurement apparatus comprising:
the ultrasonic probe according to claim 5 that performs transmission and reception of ultrasonic waves toward the same blood vessel of a subject in parallel; and
the control device according to claim 7,
wherein the measurement apparatus measures vasculature biological information.

19. The measurement apparatus according to claim 9,
wherein at least one of pulse wave propagation velocity, a blood vessel diameter, and a blood flow rate is measured as the vasculature biological information.

20. The measurement apparatus according to claim 9, further comprising:
a relative blood vessel position determination unit that determines relative blood vessel positions which are blood vessel positions as measurement targets in the respective ultrasonic transducer arrays, on the basis of the individual received signal;
a propagation length calculation unit that calculates a propagation length of a pulse wave along a blood vessel by using a gap between the adjacent ultrasonic transducer arrays and a gap between the relative blood vessel positions; and
a pulse wave propagation velocity calculation unit that calculates pulse wave propagation velocity by using the individual received signal and the propagation length.
